(19) United States
(12) Patent Application Publication
(10) Pub. No.: US 2009/0226903 A1
(43) Pub. Date: Sep. 10, 2009

(54) Thermal Cycler

(75) Inventor: Ben Cobb, Leatherhead (GB)

Correspondence Address: WOLF GREENFIELD & SACKS, P.C., 600 ATLANTIC AVENUE, BOSTON, MA, 02210-2206, US

(73) Assignee: Evogen Ltd., Surrey (GB)

(21) Appl. No.: 12/084,796

(22) PCT Filed: Nov. 13, 2006
(86) PCT No.: PCT/GB2006/050385

§ 371 Date: Apr. 17, 2009

(30) Foreign Application Priority Data
Nov. 11, 2005 (GB) 0522972.9

(51) Int. Cl. C12Q 1/68; C12M 1/00; C12P 19/34

(52) U.S. Cl. 435/6; 435/303.1; 435/287.2; 435/91.2

(57) ABSTRACT

A thermal cycling device comprising a casing defining an air inlet and an air outlet, and a heater for heating air within the casing. A sample is located within the casing, and a selectively positionable member within the casing may be moved so as to either direct cool air from the inlet or warmed air over the sample. In this way, a sample may be repeatedly heated and cooled primarily using air movements. The heater is separated from the cool air flow, such that the heater may be maintained at a constant temperature. This avoids the need to heat or cool the heater, while the mixing of warm and cool air allows for relatively rapid cycling.

### FIELD OF THE INVENTION

The present invention relates to a thermal cycler apparatus, for use in thermal cycling reactions. Aspects of the invention relate to methods for performing thermal cycling reactions.

### BACKGROUND TO THE INVENTION

Thermal cycling applications are an integral part of contemporary molecular biology. For example, the polymerase chain reaction (PCR), which is used to amplify nucleic acids, uses a series of DNA melting, annealing, and polymerisation steps at different temperatures to greatly amplify the amount of DNA in a sample. Other thermal cycling applications are also known.

A typical thermal cycling apparatus consists of a metal block containing an appropriate number of recesses to receive one or more reaction vessels. The block may be shaped to conform to a 96-well plate format. The metal block acts as a heating and cooling element, usually as a Peltier thermal cycler. The apparatus will also include a fan or the like, to assist in heat transfer from the metal block.

Although convenient, such thermal cyclers suffer from a number of disadvantages. Key among these is the thermal mass of the block itself; since this must be heated or cooled to the desired temperature before the reaction vessels will reach the desired temperature, there is a significant lag effect in the heating and cooling, leading to longer cycling times. Further, additional energy must be used to heat or cool the metal block, leading to inefficiencies in the process.

Typically, a thermal cycling reaction will be carried out in the apparatus until finished, and the reaction vessel then removed and analysed to ensure that the reaction has worked. For example, it is known to incorporate labelled nucleotides into a reaction which may later be detected in amplified nucleic acids. Certain thermal cyclers may include detectors which can be used to detect incorporated label during the progress of a thermal cycling reaction (known as Real Time PCR). However, it can be difficult to include a suitable detector system within a conventional thermal cycler, owing to the restricted space within which the reaction vessels are maintained to ensure efficient thermal transfer.

It is among the objects of embodiments of the present invention to provide an alternative thermal cycler. Certain embodiments of the invention are intended to provide an alternative cycler including a detector for monitoring progress of a cycling reaction.

### SUMMARY OF THE INVENTION

According to a first aspect of the present invention, there is provided a thermal cycler comprising:

a casing defining an air inlet and an air outlet;

means for moving air within the casing;

a sample holder for receiving a sample;

heating means for heating air within the cycler; and

a first selectively positionable member for modifying an air flow path within the casing, such that in a first position, an air flow path is defined between the air inlet and the sample holder; and in a second position, an air flow path is defined between the heating means and the sample holder, while air flow is restricted or prevented between the air inlet and the sample holder.

In use, therefore, the present invention may be used to heat and cool a sample using air flowing along one or other of the flow paths. With the member in the first position, cool air may flow from the air inlet to the sample holder to cool a sample therein. With the member in the second position, cool air is restricted or prevented from flowing from the inlet to the sample, and heated air is able to flow from the heating means to the sample, in order to heat the sample. The use of air, rather than a metal block or similar in contact with the sample, means that heating and cooling is not slowed by the thermal mass of the block, improving rate of temperature change. The separation of the heater from the cool air means that the heater does not need to be cooled as part of the cooling process, but can be maintained in a heated state throughout. This not only improves response time when switching to cool air, but also improves heating time, since heated air will be rapidly available when the member is repositioned.

Preferably when the member is in the second position air recirculates between the heating means and the sample holder. Recirculation of heated air improves efficiency and increases the heating rate. Recirculation of hot air gives greater tolerance in hot air control, providing a more uniform temperature at the sample holder, and between cycles.

Preferably when the member is in the second position air flow is substantially prevented between the air inlet and sample holder. This serves to minimise unwanted mixing of cooler air from the inlet with warmer air from the heating means.

When the member is in the first position, preferably the air flow path continues from the sample holder to the heating means. This ensures that there is a supply of air passing the heating means to avoid overheating of the heating means, and also for rapid use when the member moves from the first to the second position. Preferably also the air flow path continues from the heating means to the outlet.

The cycler may further comprise a second selectively positionable member for modifying an air flow path within the casing, such that in a first position the second member restricts or prevents air flowing from the heating means to the outlet, while in the second position the second member permits air to flow from the heating means to the outlet. In the first position, this arrangement will encourage recirculation of heated air between the heating means and the sample holder (when the first member is in an appropriate position), while the second position allows venting of unwanted heated air. Preferably in the first position the second member substantially prevents air flowing from the heating means to the outlet.

In preferred embodiments, the first member has an intermediate third position, in which air flow paths exist both between the air inlet and the sample holder, and between the heating means and the sample holder. This arrangement allows cool air from the inlet to mix with warm air from the heating means before passing the sample holder, allowing intermediate temperatures of the sample to be reached or maintained. Conveniently the first member has a plurality of intermediate positions, whereby the relative proportions of cool and warm air may be adjusted in order to regulate the temperature of the mixed air.

Certain embodiments of the invention may further comprise cooling means disposed adjacent the air inlet. This allows indrawn air to be cooled to a desired temperature before reaching the sample, and provides for more precise temperature regulation.

Preferably the heating means comprises a heating element; conveniently an electrical heating element, such as a wire-wound heating element. Such an element has advantages over a Peltier-effect device in that its lower thermal mass allows the heater to rapidly reach its operating temperature, and so to rapidly heat air in the vicinity. Two or more heating means may be provided, depending on requirements for heating. Preferably the heating means is adjustable, such that it may be operated at different power levels depending on heating requirements. The heating means may incorporate a thermoregulator, to maintain its temperature at a desired level.

The heating means may be an elongate heater, having first and second ends. In preferred embodiments, the regions adjacent the ends of the heater are not active in heating; preferably also these regions maintain a generally uniform air flow profile compared with the active regions of the heater. This may be described as the “effective length” of the heater being less than the “actual length”. Where the heater comprises a heating element, a portion of the element may be inactive. For example, the heating element may be a wire-wound heating element, and the windings adjacent the ends of the heater may be short-circuited such that they are inactive. The use of inactive windings, rather than no windings, maintains a generally uniform air flow profile across the heater, which is useful in controlling the temperature profile within the device.

The heating means may comprise a plurality of heating elements; preferably at least 2, 3, or 4 heating elements. In preferred embodiments, at least two of the elements have different “effective lengths” for heating, as described above. For example, where four elements are present, two have a shorter effective length than the other two. This arrangement may be used to compensate for heating of the casing of the device. The power to the elements may be adjusted to perform this compensation; the inventors have noted that providing higher power to an element not only increases the heat output from the element, but also increases the distance beyond the element for which air is heated—that is, the “effective length” of the heater is increased. By adjusting the ratio of power to the elements of longer and shorter effective length, a desired overall heating profile and effective length may be obtained. Thus, when the casing is cool, the elements are controlled to provide a greater effective length, which will heat the casing; and when the casing is warm, a lesser effective length is used, as the casing will provide some heating to the air.

The thermoregulator may comprise a plurality of temperature sensors, for example thermocouples. A first sensor may be located towards the centre of the heater, and a second sensor located towards the end of the heater. By monitoring the temperature difference between these sensors, the desired ratio of power to longer and shorter heating elements may be determined to obtain a specified effective length of the heaters.

The means for moving air conveniently comprises a fan; preferably a centrifugal fan. The means for moving air is preferably located within the casing, and more preferably along one of the air flow paths. In a preferred embodiment, the means for moving air is located along the air flow path between the sample holder and the heating means; in certain embodiments, the air flow path from the sample holder to the heating means is distinct from the flow path from the heating means to the sample holder, and the means for moving air is located along the flow path from the sample holder to the heating means.

The first (and preferably second) selectively positionable members are conveniently vanes. The members may be operatively associated with motor means for changing the position of the members; conveniently a stepper motor or the like. Alternatively, a solenoid or other arrangement may be used.

Preferably the sample holder is for receiving a sample reaction vessel. The reaction vessel may be in the form of an elongate tube or similar; conveniently the vessel may be a thin-walled tube, or the like. The sample holder is preferably arranged so as to expose a substantial portion of the reaction vessel to air; for example, the sample holder may support the vessel by a limited portion such as the neck of an elongate tube. Conveniently the sample holder is for receiving a plurality of reaction vessels. Preferably the sample holder is elongate, and defines a plurality of reaction vessel receiving portions arranged linearly. Preferably the sample holder is arranged to hold a reaction vessel at an angle other than vertical; this allows the reaction vessel to act as a vane for promoting mixing of air within the cycler.

Preferably the sample holder is located within a volume for receiving sample vessels. The volume may be of a corresponding shape to the sample holder, and preferably is a generally elongate volume for receiving a plurality of reaction vessels arranged in a linear manner. The use of an elongate volume allows an increased surface area:volume ratio, which may be heated or cooled more rapidly by replacement of air therein than volumes with lower surface are: volume ratios.

In embodiments where the sample holder and volume are elongate, it is preferred that any or all of the heating means, air moving means, and selectively positionable members, are similarly elongate. This ensures a more even heat distribution and transfer, as well as more even air movement.

Preferably the cycler further comprises at least one temperature sensor to monitor temperature within the cycler. Conveniently the cycler comprises temperature sensors to monitor temperature at the sample holder at least. Other monitoring locations may also be used. The cycler may further comprise means for recording monitored temperatures, and/or means for displaying monitored temperatures to a user. The monitored temperatures may be used to regulate the heating means and/or the selectively positionable members in order to achieve a desired temperature.

Preferably the cycler further comprises a control system to activate any or all of the first and/or second selectively positionable members, the means for moving air, and the heating means in accordance with a desired programmable temperature profile. The control system may use the temperature sensor to cycle the temperature of the samples contained within the sample reaction vessels using air flow and heater power control.

In a preferred embodiment, the control system comprises microcontroller electronics that provide standalone control of the instrument, and temperature control of the samples. The controller may also provide data output according to light sensor outputs from the apparatus.

In embodiments where temperature uniformity of the samples is required within defined limits, a further control system with additional heating elements may provide active compensation to temperature uniformity errors as detected by additional temperature sensors. The preferred embodiment implements three additional heaters with their respective temperature sensor and control systems.

Preferably the cycler further comprises a light sensor, and preferably also a light source. This allows the cycler to be used in detecting fluorescence or other signal from a reaction as the reaction proceeds in real time. The light source and light sensor may encompass any electromagnetic radiation, not merely visible light. Preferably the light sensor is disposed to one side of the sample holder, such that the sensor will in use be located to one side of a sample in the cycler. Preferably also the light source is located on the same side of the sample holder. Conveniently the light source is arranged at around 45° to the light sensor. Illuminating the sample from the side allows a truncated optics path while maximising the viewing area, thereby increasing sensitivity and reducing tolerance errors. Locating both source and sensor on the same side of the sample reduces leakage from the source to the sensor, ensuring that only reflected light or fluorescence will be detected. A relative angle of 45° between the source and detector avoids illuminated light breaking through into fluorescent output. Illuminated light also reflects away from the detector. The cycler may also comprise filters to restrict light of particular wavelengths; where the desired angle is used, imperfect filters may be used, thereby lowering cost of production. The cycler may further comprise a second light source; the second source may also be at 45° to the detector without interfering with the first source. This allows for a relatively low-cost two-label detection system, where the two sources illuminate at different wavelengths. Where the cycler is intended to use a plurality of samples or reaction vessels in each cycling reaction, the cycler may conveniently comprise at least one light source/sensor combination for each sample.

In preferred embodiments of the invention, the light source is an LED or similar, while the light sensor is a photodiode or the like. The sensor is conveniently a log-response detector, which allows for a wider dynamic range, and a wider copy number of nucleic acids which can be detected. This arrangement allows for simple, robust components to be used without the requirement for lenses or complex optics arrangements. Such a source/sensor combination has been found to be sufficient to obtain qualitative information on the progress of a reaction (for example, that amplification is occurring). For many applications, such data is sufficient, and it is not necessary to quantitate the progress of the reaction. The use of an LED/photodiode arrangement also reduces the need for critical positioning or distancing of the source and sensor with respect to the sample, again thereby making the cycler more robust. Conveniently the source and sensor operate at different wavelengths of light; for example, a preferred source is an LED emitting light at 490 nm, while a preferred sensor is most sensitive to light at 530 μm. This is consistent with typical fluorophores used in biochemical reactions. Modulated illumination of the LED or LEDs can be used, in order to help remove noise and background from the signal.

The cycler may further comprise a computer processor, which may be used for monitoring and controlling the light source and detector, temperature regulation, the cycling program, and the like. The processor is conveniently user-programmable, to allow selection of appropriate cycling programs for particular reactions.

A further aspect of the present invention provides a thermal cycler comprising:

a casing;

a sample holder for receiving a sample;

means for heating and cooling a sample within the sample holder; and

a light sensor and a light source located to one side of the sample holder, wherein the light source is arranged at around 45° to the light sensor.

The cycler may further comprise a second light source; the second source may also be at 45° to the detector, and is preferably located to the same side of the sample holder as the first source.

In preferred embodiments of the invention, the light source is an LED or similar, while the light sensor is a photodiode or the like. The sensor is conveniently a log-response detector.

A further aspect of the present invention provides a method of performing a thermal cycling reaction, the method comprising the steps of:

a) operating an air heater to maintain a reservoir of heated air within a volume;

b) heating a sample by passing heated air from the reservoir over the sample;

c) cooling said sample by passing cool air from outside the reservoir over the sample; and

d) repeating steps b) and c) a plurality of times to alternately heat and cool the sample.

The method may further comprise the step of passing cool air from the sample to the reservoir of heated air.

The method may also comprise the step of mixing heated air from the reservoir and cooled air from outside the reservoir, and passing the mixed air over the sample. This may be used either to heat or cool the sample more slowly, or may be used to maintain the sample at a desired temperature. This step may be performed and repeated along with steps b) and c).

Heated air may also be periodically vented from the reservoir, to prevent overheating.

The method may farther comprise the step of illuminating the sample with a particular wavelength of light, and detecting emitted light from the sample.

The present invention also provides a method of operating a thermal cycler, the method comprising the steps of:

placing a first selectively positionable member in a first position to define an air flow path between an air inlet and a sample holder;

passing cool air from the air inlet over the sample holder to cool a sample therein;

placing the first selectively positionable member in a second position to define an air flow path between an air heater and the sample holder, and to restrict or prevent air flow between the air inlet and the sample holder;

passing heated air from the heater over the sample holder to heat a sample therein; and

repeating the above steps to thermally cycle the sample.

The method may further comprise the step of placing the first selectively positionable member in an intermediate position, to allow heated air and cool air to mix, and passing the mixed air over the sample holder.

The method may also comprise the step of placing a second selectively positionable member in a first position to restrict or prevents air flow from the heater to an air outlet. The method may further comprise the step of placing the second selectively positionable member in a second position to permit air to flow from the heater to an air outlet.

The method may further comprise the step of illuminating the sample with a particular wavelength of light, and detecting emitted light from the sample.

### BRIEF SUMMARY OF THE DRAWINGS

These and other aspects of the present invention will now be described by way of example only and with reference to the accompanying drawings, in which:

FIG. 1 shows a side sectional view of a thermal cycler in accordance with an embodiment of the present invention;

FIG. 2 shows a top sectional view of the cycler of FIG. 1;

FIG. 3 shows a front sectional view of the cycler of FIG. 1;

FIG. 4 shows a schematic side view of the cycler of FIG. 1;

FIGS. 5, 6, and 7 show schematic views of different stages in the operation of the cycler of FIG. 4;

FIG. 8 is a graph showing the temperature within the cycler of FIG. 1 over a number of cycles;

FIG. 9 shows a side view of the cycler illustrating additional heaters;

FIG. 10 shows a plan view of the cycler of FIG. 9, illustrating the heaters; and

FIG. 11 shows a perspective view of a heater which may be used with embodiments of the present invention.

### DETAILED DESCRIPTION OF THE DRAWINGS

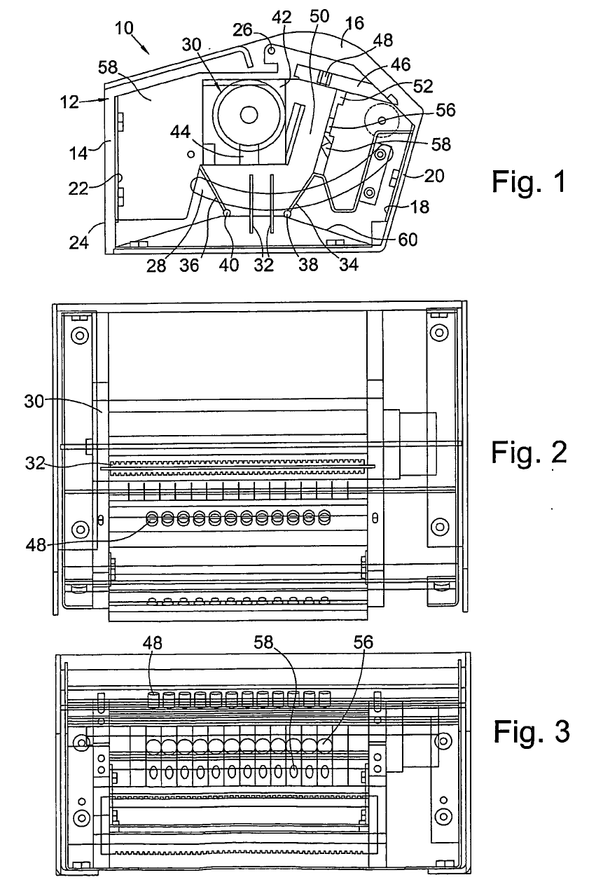

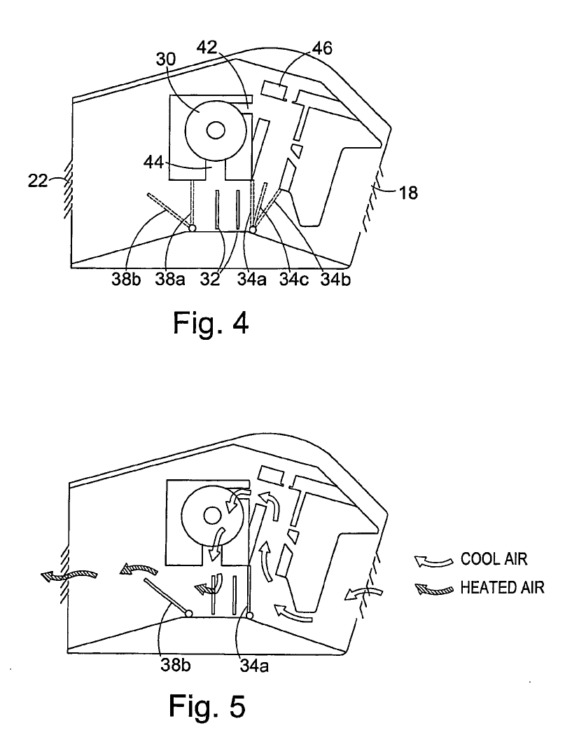

FIGS. 1 to 3 show side, top, and front sectional views of a thermal cycler in accordance with an embodiment of the present invention. For clarity in the following description, reference may usefully also be made to the simplified diagram of FIG. 4.

The thermal cycler 10 of the present invention includes a casing 12 which forms a body part 14 and a lid part 16. The front portion of the body part 14 includes an opening which forms an air inlet 18, which is covered by a louvred panel 20. A similar air outlet 22 and louvred cover 24 are located on the rear portion of the body part 14.

The lid part 16 of the cycler is mounted on a pivot point 26, with a cam (not shown) running within a curved guideway 28 within the body of the cycler. This allows the lid part 16 to be pivoted open to allow access to the interior of the cycler, while the cam and guideway ensure smooth limited movement of the lid.

Within the body of the cycler are located a centrifugal fan 30, a pair of wire wound heaters 32, and first and second movable vanes 34, 36, mounted on respective pivot points 38, 40. The centrifugal fan 30 has an air inlet 42 and an air outlet 44. The air outlet 44 is adjacent the wire wound heaters 32 and the movable vanes 34, 36. A baffle 60 extends across the lower part of the casing, and contacts the pivot points 38, 40 to restrict air movement in the lower part of the casing.

Adjacent the fan air inlet 42 is a sample holder 46, which serves to hold reaction vessels containing suitable reaction mix within the cycler. The holder 46 is elongate, having in this embodiment twelve linearly-arranged openings 48 each of which is suitable for receiving a reaction vessel. The openings 48 of the holder receive reaction vessels by the neck, such that the bulk of the vessel will extend below the holder 46 into a similarly elongate volume 50, extending a substantial portion of the length of the cycler. The fan 30, heaters 32, and movable vanes 34, 36 are all also similarly elongate, and extend substantially along the length of the sample holder.

Mounted below the sample holder 46 is an optics block 52, within which is mounted an LED 54 and a photodiode 56 for each reaction vessel. The optics block 52 is intended to hold the LED 54 and photodiode 56 adjacent the side of a reaction vessel within the sample holder 46, such that the LED may illuminate the reaction vessel, and the photodiode detect light emitted from the sample. The LED 54 is directed at a 45° angle to the photodiode.

Not shown in these Figures are the controlling electronics for the optics block, fan, heaters and movable vanes; these may be located in the rear section 58 of the cycler.

The operation of the cycler will now be described, with particular reference to FIGS. 4 to 7. Referring first of all to FIG. 4, this simplified diagram shows a number of key components of the cycler:the air inlet 18 and outlet 22, the sample holder 46, the fan 30 with fan inlet 42 and outlet 44, and the heaters 32. Also shown in FIG. 4 are the first and second movable vanes 34, 38. The Figure illustrates three possible positions of the first vane 34, shown in dotted lines: a first position 34a, in which the vane contacts a lower portion of the housing of the fan 30 to form an air flow path between the inlet 18 and the sample holder 46; a second position 34b, in which the vane contacts a lower portion of the sample holder 46 to block this air flow path, and to create an air flow path between the heaters 32 and the sample holder 46; and a third intermediate position 34c, in which both air flow paths are open.

Similarly, there are two possible positions of the second vane; a first position 38a in which the vane contacts a lower portion of the housing of the fan 30 to block an air flow path between the heaters and the air outlet 22; and a second position 38b in which the vane allows air to flow between the heaters 32 and the air outlet 22.

In addition to the selectable air flow paths adjusted by movement of the first and second vanes, the cycler is arranged such that the fan 30 will draw air from the sample holder 46 into the fan inlet 42, and will expel air from the fan outlet 44 to the heaters 32.

The positions of each of the vanes 34, 38 are controlled by a stepper motor arrangement (not shown). By adjusting the positions of the vanes 34, 38, the cycler may be used to alternately heat and cool a sample in the sample holder, as will be described.

FIGS. 5 to 7 show the cycler in cooling, rapid heating, and slow heating mode respectively. In each figure, the pattern of air flow is indicated by arrows; shaded arrows indicate heated air, with unshaded arrows indicating cool air. It should be noted that during each operating mode, the heaters 32 are being operated to heat air adjacent the heaters, and the fan 30 is being operated to circulate air along the allowable air flow paths. The continual operation of both the fan and the heaters means that there is no time lag while the heater is itself warmed or cooled, or while the fan is brought to speed, when a switch in operating mode is needed. This in turn means that cycles may be faster than conventional thermal cyclers, leading to reduced overall time for cycling reactions, and a faster response when switching from heating to cooling. Removal of the need to heat or cool the heater itself leads to improved energy efficiency.

FIG. 5 shows the cycler in cooling mode, with the first vane in the first position 34a, and the second vane in the second position 38b. Cool air is drawn from the outside through the air inlet 18 and past the sample holder 46, to thereby cool a reaction vessel held by the holder. The cool air continues through the fan 30, and from the fan outlet 44 over the heaters 32. The heated air is then expelled from the cycler via the air outlet 22, and is not used to alter the temperature of the reaction vessel. Continual operation of the heaters 32 serves to maintain a reservoir of hot air adjacent the heaters, while continual inflow of cool air to the heaters prevents overheating. The reservoir can thus be maintained at a constant high temperature for use when needed.

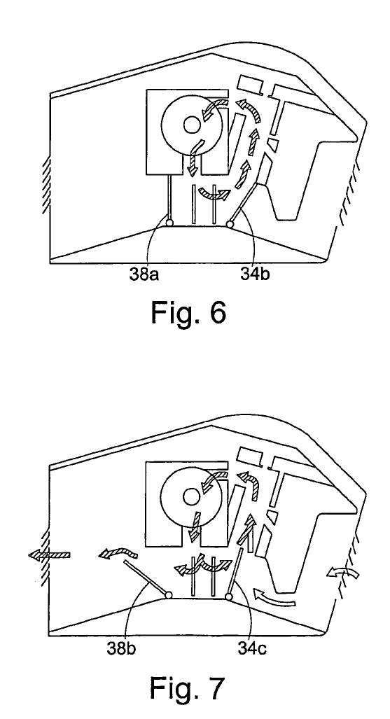

When the cycler is used in rapid heating mode, the vanes are set as shown in FIG. 6. The first vane is in the second position 34b, and the second vane is in the first position 38a. In this mode, cool air is no longer drawn in from outside the cycler. Instead, preheated air is allowed to flow from the heaters 32 past the sample holder 46, to heat a sample held therein. The heated air then is recirculated through the fan, and past the heaters once more. This recirculation maintains the air at a relatively high temperature, and serves to rapidly raise the temperature of the sample.

An alternative heating mode is illustrated in FIG. 7, which allows for slower heating of the sample, or for maintenance of the sample at a lower temperature. In this mode, the first vane is in an intermediate position 34c, while the second vane is in the second position 38b, allowing venting of heated air to outside. The intermediate position 34c of the first vane allows some cool air to be drawn in from outside and pass over the sample holder, while also allowing heated air to flow from the heaters to the sample holder. In this mode, a mixture of heated and cool air flows over the sample, so ramping its temperature more slowly than either of the first two modes. This mode may also be used to maintain temperature at a desired level, or may be used to finely adjust the temperature of the sample, by altering the relative mixture of heated and cool air. Of course, the temperature of the heaters themselves may also be adjusted to alter the temperature of the heated air, but that approach reintroduces some thermal lag of the heater into the cycling process. This mode of operation is particularly preferred when it is desired to implement a slow but accurately controlled temperature ramp, for example as required for a melt process.

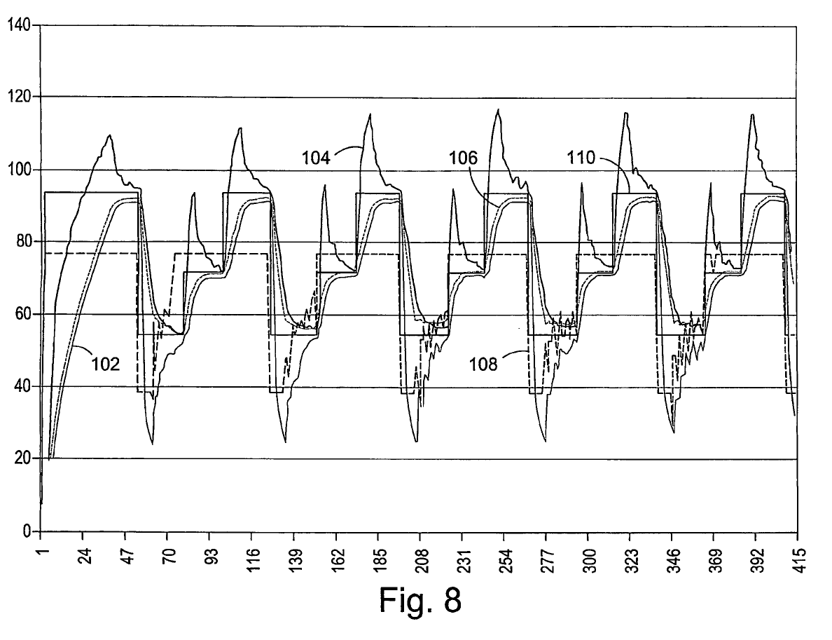

FIG. 8 shows a graph of the temperature within the cycler of FIG. 1 at different locations over a number of cycles. The X axis indicates time in seconds, while the Y axis shows temperature in degrees centigrade. Line 102 shows the actual reaction vessel temperature; it can be seen that this temperature begins by ramping from 20° C. to 91° C. at around 43 seconds. A number of cycles then follow, with the reaction vessel regularly cycling between 91° C. for around 13 seconds, before falling to 55° C. within 20 seconds to be maintained at that temperature for 8 seconds. The temperature is then ramped to 70° C. in 5 seconds, and maintained at 70° C. for 16 seconds, before returning to 91° C. in 10 seconds. This cycling is repeated a number of times, and it is apparent that the temperature of the reaction vessel is very accurately maintained, and rapidly ramps from one temperature to the next. Further, the temperature is consistent between cycles.

Line 104 shows the air temperature adjacent the reaction vessel; although this temperature apparently overshoots the desired reaction vessel temperature, it rapidly resolves to the desired temperature.

Line 106 shows the calculated sample temperature within the reaction vessel, based on the reaction vessel temperature and the air temperature. This closely conforms to the reaction vessel temperature, but is slightly more accurate.

Line 108 does not show temperature, but shows the vane position of the first vane 34; this switches between a first position (lower portion of the curve), when the reaction vessel is cooling, and a second position (upper portion of the curve), when the reaction vessel is heating.

Finally, curve 110 is a theoretical ideal curve showing the ideal sample temperature with instantaneous transition between temperatures. It can be seen that the actual temperature closely approximates this.

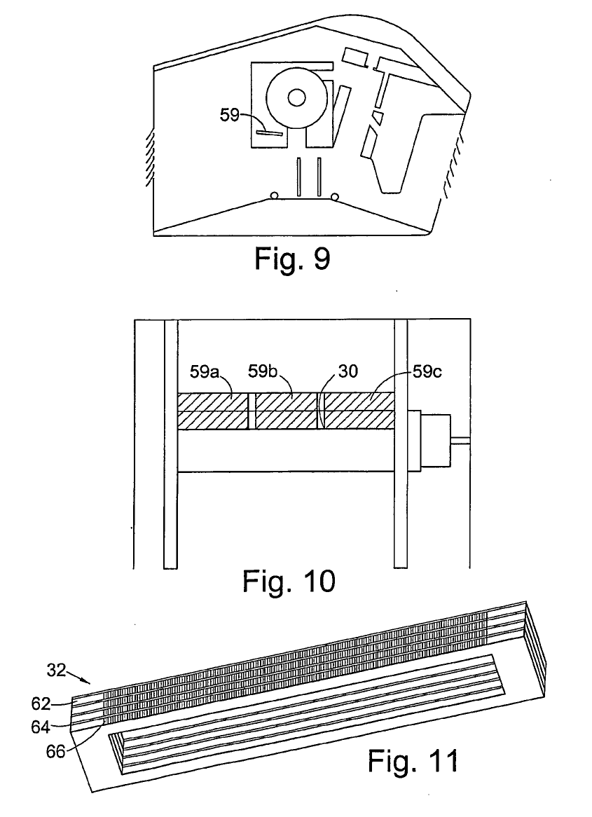

FIG. 9 shows a side view of the general location of additional heaters 59 spaced at intervals across the airflow, activated by the microcontroller according to algorithms in response to additional temperature sensors. The additional heaters 59 may be used to compensate for small temperature uniformity errors that are introduced between the plurality of reaction vessels. This compensation technique enhances the sample temperature uniformity.

FIG. 10 shows a plan view of the apparatus of FIG. 9, illustrating three additional heater elements 59a, 59b and 59c used for temperature uniformity control.

FIG. 11 shows a perspective view of a heater 32 which may be used with embodiments of the invention. The basic construction comprises four formers 62 fabricated from silvered mica. Each former is 3 mm thick and is fabricated as a hollow rectangle that is notched on each of its long dimensions at intervals for the purpose of spacing the element wire. The heater also comprises five spacers 64 having the same shape as the formers but without the notches and being only 1 mm thick. These spacers are interposed between the four main formers and also one on either face of the element so that the order is spacer-former-spacer-former-spacer-former-spacer-former-spacer.

Element wire (not shown) is wrapped around each of the formers to produce an element. The notches 66 are spaced at 3 mm intervals and the element wire used is of 35 ohm impedance and has a circular cross section. It will be apparent that the details of the former, spacer, and wire construction may be varied as appropriate. If the element wire extends the full length of this former we have found that the sides of the instrument may become hot on account of the heat profile from the element extending beyond the termination of the windings. In practise this means that the air passing through the element becomes hotter at the edges of the instrument than in the middle because it has the contribution from the active element as well as the stored heat energy in the instrument walls.

In view of this we attempted to reduce the active length of the element by removing windings from either end—in this way we hoped to prevent the instrument walls from heating quite so much and thereby achieve a more uniform air temperature across the full width of the instrument. However we found that physically removing windings from the element had a detrimental effect on the thermal uniformity. The lack of windings at the edge of the instrument changed the air flow characteristics—the air was able to preferentially flow around the element rather than maintain laminar flow across the width of the instrument. This meant in practise that at the edge of the element the air temperature either peaked or exhibited a minimum—which was even more unacceptable than had been the case with a single long element when the only problem had been increased temperature at the sides of the instrument.

To solve this problem we decided to leave the windings in place for the full width of the element so that the air flow faced exactly the same flow resistance across the full width of the instrument, but we applied an electrical short circuit to the windings at the edge of the instrument so that they were in fact inactive and did not heat up because no current was flowing through them. We achieved this by applying a short length of copper tape to each end of the spacer adjacent to any former for whose element we wished to shorten the active length. This inclusion of inactive windings proved beneficial to the operation of the heater assembly and successfully overcame the edge problem that arose with the physically shorter elements.

Having established a successful means of reducing the effective length of the element it soon became clear that, in some circumstances, the instrument walls heated up over the period of operation such that an appropriate length of element at the start of operation was too long by the end of operation. However we had noted that increasing the power in any element did not merely increase the temperature in the middle of the instrument, it also increased the distance beyond the end of the element for which air was heated by the element. We amended two of the elements to be shorter than would be appropriate for thermal uniformity—even when the instrument walls are at maximum temperature. We left the other two elements at full length. The overall effect of this was to achieve an effective distance over which the air temperature was uniform that was longer than the shorter elements in the heater assembly. By modifying the ratio of power input to the longer and shorter elements it proved possible to adjust the length of this effective distance over which the air temperature was maintained uniform.

This heater structure comprising a combination of longer and shorter elements allowed the use of a control algorithm to dynamically maintain thermal uniformity over the width of the instrument. Having identified the technical basis for adjusting the length of the thermally uniform portion of the instrument, we had now a tool by which to compensate for changes in the instrument temperature. When the walls of the instrument are cool it is appropriate to have a longer section of uniformly warm air—but as the walls of the instrument heat up during operation it is more appropriate to have a slightly shorter section of uniformly warmed air because the walls of the instrument contribute an increased effect on the air temperature at the sides of the instrument.

We positioned a thermocouple at the mid point of the instrument width and a second thermocouple at the edge of the instrument width. By monitoring these two temperatures in parallel we were able to establish the degree of imbalance in the air temperature across the instrument width and use this as a control measure to direct the effective length of the “dynamic” element comprised of the two electrically-short and two electrically-long physical elements. In fact our control algorithm needed to take account of the fact that increasing power to the “long” element added heat to the middle as much as to the edges of the instrument so as power was increased in the “long” element our control algorithm needed to correspondingly reduce power to the “short” element. This balancing of power was achieved by modulating the width of the voltage pulse supplying power into the elements.

The present invention therefore provides a simple, robust cycling mechanism which allows for rapid and accurate thermal cycling using heated or cool air. This mechanism allows for a more efficient cycling than prior art devices, since the heating mechanism does not need to be heated and cooled in each cycle. The foregoing description is for illustrative purposes only, and the skilled person will envisage a number of modifications which may be made to the described arrangement while remaining within the scope of the invention. For example, the illustrated airflow path may be modified if desired; in particular, the flow of warm and cool air may be reversed such that the inlet and outlet functions are altered. Suitable alterations to the operations of the vanes may also be made.

1. A thermal cycler comprising:
a casing defining an air inlet and an air outlet;
means for moving air within the casing;
a sample holder for receiving a sample;
heating means for heating air within the cycler; and
a first selectively positionable member for modifying an air flow path within the casing, such that in a first position, an air flow path is defined between the air inlet and the sample holder; and in a second position, an air flow path is defined between the heating means and the sample holder, while air flow is restricted or prevented between the air inlet and the sample holder.

2. The cycler of claim 1, wherein when the member is in the second position air recirculates between the heating means and the sample holder.

3. The cycler of claim 1, wherein when the member is in the second position air flow is substantially prevented between the air inlet and sample holder.

4. The cycler of claim 1, wherein when the member is in the first position, the air flow path continues from the sample holder to the heating means.

5. The cycler of claim 4, wherein when the member is in the first position, the air flow path continues from the heating means to the outlet.

6. The cycler of claim 1 further comprising a second selectively positionable member for modifying an air flow path within the casing, such that in a first position the second member restricts or prevents air flowing from the heating means to the outlet, while in the second position the second member permits air to flow from the heating means to the outlet.

7. The cycler of claim 6, wherein in the first position the second member substantially prevents air flowing from the heating means to the outlet.

8. The cycler of claim 1, wherein the first member has an intermediate third position, in which air flow paths exist both between the air inlet and the sample holder, and between the heating means and the sample holder.

9. The cycler of claim 8, wherein the first member has a plurality of intermediate positions, whereby the relative proportions of cool and warm air may be adjusted in order to regulate the temperature of the mixed air.

10. The cycler of claim 1, further comprising cooling means disposed adjacent the air inlet.

11. The cycler of claim 1, wherein the heating means comprises a heating element.

12. The cycler of claim 11, wherein the heating element is a wire-wound heating element.

13. The cycler of claim 1, wherein the heating means is an elongate heater having first and second ends, wherein the regions adjacent the ends of the heater are not active in heating.

14. The cycler of claim 13, wherein the regions adjacent the ends of the heater maintain a generally uniform air flow profile compared with the active regions of the heater.

15. The cycler of claim 13, wherein the heater comprises a heating element, a portion of which is inactive.

16. The cycler of 1, wherein the heating means comprises a plurality of heating elements.

17. The cycler of claim 16, wherein at least four heating elements are present.

18. The cycler of claim 16, wherein the elements are elongate having first and second ends, and the regions adjacent the ends of at least one of the elements are not active in heating.

19. The cycler of claim 18, wherein at least two of the elements have different lengths of element active in heating.

20. The cycler of claim 19, comprising means for adjusting the ratio of power supplied to said elements having different lengths of active element.

21. The cycler of claim 1, wherein the means for moving air comprises a centrifugal fan.

22. The cycler of claim 1, wherein the sample holder is arranged so as to expose a substantial portion of a held reaction vessel to air.

23. The cycler of claim 1, wherein the sample holder is for receiving a plurality of reaction vessels.

24. The cycler of claim 1, wherein the sample holder is located within a volume for receiving reaction vessels.

25. The cycler of claim 24, wherein the volume is a generally elongate volume for receiving a plurality of reaction vessels arranged in a linear manner.

26. The cycler of claim 1, further comprising at least one temperature sensor to monitor temperature within the cycler.

27. The cycler of claim 1, further comprising a light sensor and a light source.

28. The cycler of claim 27, wherein the light sensor and light source are disposed to the same side of the sample holder.

29. The cycler of claim 27, wherein the light source is arranged at around 45° to the light sensor.

30. The cycler of claim 27, further comprising a second light source arranged at 45° to the detector.

31. A thermal cycler comprising:
a casing;
a sample holder for receiving a sample;
means for heating and cooling a sample within the sample holder; and
a light sensor and a light source located to one side of the sample holder, wherein the light source is arranged at around 45° to the light sensor,

32. The cycler of claim 31, further comprising a second light source arranged at 45° to the detector.

33. A method of performing a thermal cycling reaction, the method comprising the steps of:
a) operating an air heater to maintain a reservoir of heated air within a volume;
b) heating a sample by passing heated air from the reservoir over the sample;
c) cooling said sample by passing cool air from outside the reservoir over the sample; and
d) repeating steps b) and c) a plurality of times to alternately heat and cool the sample.

34. The method of claim 33, further comprising the step of passing cool air from the sample to the reservoir of heated air.

35. The method of claim 33, further comprising the step of mixing heated air from the reservoir and cooled air from outside the reservoir, and passing the mixed air over the sample.

36. The method of claim 33, further comprising the step of illuminating the sample with a particular wavelength of light, and detecting emitted light from the sample.

37. A method of operating a thermal cycler, the method comprising the steps of:
placing a first selectively positionable member in a first position to define an air flow path between an air inlet and a sample holder;
passing cool air from the air inlet over the sample holder to cool a sample therein;
placing the first selectively positionable member in a second position to define an air flow path between an air heater and the sample holder, and to restrict or prevent air flow between the air inlet and the sample holder;
passing heated air from the heater over the sample holder to heat a sample therein; and
repeating the above steps to thermally cycle the sample.

38. The method of claim 37, further comprising the step of placing the first selectively positionable member in an intermediate position, to allow heated air and cool air to mix, and passing the mixed air over the sample holder.

39. The method of claim 37, further comprising the step of placing a second selectively positionable member in a first position to restrict or prevent air flow from the heater to an air outlet.

40. The method of claim 39, further comprising the step of placing the second selectively positionable member in a second position to permit air to flow from the heater to an air outlet.

41. The method of claim 37, further comprising the step of illuminating the sample with a particular wavelength of light, and detecting emitted light from the sample.

42. The cycler of claim 1, further comprising one or more additional heating elements spaced across the airflow path.
